(19) United States
(12) Patent Application Publication
(10) Pub. No.: US 2012/0010479 A1
(43) Pub. Date: Jan. 12, 2012

(54) METHOD, COMPUTER READABLE MEDIUM AND DEVICE FOR DETERMINING THE TEMPERATURE DISTRIBUTION IN A TISSUE

(75) Inventors: Christian Eusemann, Malvern, PA (US); Bernhard Schmidt, Furth (DE); Martin Sedlmair, Forchheim (DE)

(73) Assignee: SIEMENS AG, Munich (DE)

(21) Appl. No.: 13/179,044

(22) Filed: Jul. 8, 2011

Related U.S. Application Data
(60) Provisional application No. 61/362,951, filed on Jul. 9, 2010.

(30) Foreign Application Priority Data
Sep. 22, 2010 (DE) 10 2010 041 175.2

(51) Int. Cl. A61B 5/0205

(52) U.S. Cl. 600/301

(57) ABSTRACT

Example embodiments of the present invention relate to a method for determining the temperature distribution in a tissue to be ablated and/or in a body tissue of a patient containing the tissue to be ablated. The method includes determining at least one item of information relating to a blood flow associated with at least one of in the tissue to be ablated and in the body tissue containing the tissue to be ablated, such that the item of information originates from a perfusion measurement of at least one of the tissue to be ablated and the body tissue containing the tissue to be ablated. The method includes determining the temperature distribution in the tissue to be ablated and/or in the body tissue containing the tissue to be ablated taking into account the at least one item of information relating to the blood flow.

### PRIORITY STATEMENT

The present application hereby claims priority under U.S.C. §119(e) on U.S. Provisional Application No. 61/362,951, filed Jul. 9, 2010 and under 35 U.S.C. §119 on German patent application number DE 10 2010 041 175.2 filed Sep. 22, 2010, the entire contents of each of which are hereby incorporated herein by reference.

### FIELD

At least one embodiment of the present invention relates to a method for determining the temperature distribution in a tissue to be ablated, for example using a probe or a needle to supply heat or cold, and/or in a body tissue of a patient containing the tissue to be ablated. Embodiments of the present invention moreover relate to a device with a computational unit for carrying out the method and to a data medium, on which a computational program implementing the method is stored.

### BACKGROUND

In medicine, ablation is becoming evermore important in tumor therapy in certain tissues or organs; here, the tumor tissue is destroyed e.g. by supplying warmth or heat or cold into the tumor tissue. Use is often made of the so-called radiofrequency ablation (RF ablation), in which RF probes or RF needles are inserted into the tumor tissue, e.g. into tumor tissue in the liver. Subsequent heating of the tips of the RF probes or the RF needles heats the tumor tissue such that the tumor cells in the tumor tissue coagulate and are finally destroyed. Alternative methods to this include e.g. cryoablation, ablation on the basis of microwaves, laser-induced thermotherapy (LITT), etc.

A problem, in each case, lies in establishing the temperature distribution in the tumor tissue and, more particularly, in the body tissue of the patient surrounding the tumor tissue, which body tissue should not be destroyed by the ablation.

### SUMMARY

At least one embodiment of the present invention are based on specifies a method, a device, and/or a data medium such that the temperature distribution in a tissue to be ablated and/or in a body tissue of a patient containing the tissue to be ablated can be determined to the best possible extent, more particularly within the scope of a simulation before carrying out the ablation.

According to at least one embodiment of the present invention, a method provides for determining the temperature distribution in a tissue to be ablated, for example using at least one probe or at least one needle to supply heat or cold, and/or in a body tissue of a patient containing the tissue to be ablated, in which the temperature distribution is determined in the tissue to be ablated and/or in the body tissue containing the tissue to be ablated taking into account at least one item of information relating to the blood flow in the tissue to be ablated and/or in the body tissue containing the tissue to be ablated, which item of information originates from a perfusion measurement of the tissue to be ablated and/or the body tissue containing the tissue to be ablated.

In order to determine the temperature distribution over time in a tissue within the scope of an ablation simulation or during an ablation, it is not sufficient to take into account only the supply of cold or heat, or the heat emission by e.g. a radiofrequency ablation probe (RF probe) or a radiofrequency ablation needle (RF needle) into the tissue to be ablated and/or into the body tissue containing or surrounding the tissue to be ablated. Rather, the blood flow and, more particularly: the dissipation of heat or cold from tissue to be ablated and/or the body tissue containing the tissue to be ablated by the blood flow is relevant because heat or cold dissipated by the blood flow no longer contributes to increasing the temperature or lowering the temperature of the tissue to be ablated and/or the body tissue surrounding the tissue to be ablated.

According to at least one example embodiment in order to take into account of the aspect of dissipating heat or cold from tissue by the blood flow when determining the temperature distribution, at least one item of information relating to the blood flow from a perfusion measurement of the tissue to be ablated and/or the body tissue containing the tissue to be ablated is obtained and to take this information into account accordingly when determining the temperature distribution. This affords the possibility of improving the simulation or estimation or determination of the temperature distribution in a tissue to be ablated and/or in a body tissue of a patient containing tissue to be ablated over time and hence the treatment or the ablation based on a simulation of the expected temperature distribution over time can be planned in an improved fashion or be accompanied or monitored in an improved fashion on the basis of an intervention-accompanying determination of the temperature distribution.

According to at least one example embodiment of the present invention, at least the flow speed of the blood of the patient flowing through the tissue to be ablated and/or the body tissue containing the tissue to be ablated and/or at least the blood volume, which flows through the tissue to be ablated and/or the body tissue containing the tissue to be ablated over time, is established within the scope of the perfusion measurement of the tissue to be ablated and/or the body tissue containing the tissue to be ablated. Here, it is particularly the flow speed of the blood flowing through the tissue to be ablated and/or the body tissue containing the tissue to be ablated that is important.

As the flow speed in the tissue to be ablated and/or in the body tissue containing the tissue to be ablated increases, so does the dissipation of heat or cold. This is particularly decisive at those points of the tissue to be ablated and/or the body tissue containing the tissue to be ablated that have relatively large blood-carrying vessels in the vicinity. It is particularly as a result of such relatively large blood-carrying vessels that the diffusion of the heat or cold or the temperature distribution around a source of heat or cold, e.g. in the form of an RF needle, deviates from the normally expected spherical form. However, by taking into account the blood flow, more particularly the flow speed of the blood, it is possible to determine the deviation and hence the actual temperature distribution around the source of heat or cold.

According to at least one example embodiment of the present invention, the temperature distribution over time in the tissue to be ablated and/or in the body tissue containing the tissue to be ablated is additionally determined on the basis of the ingress of heat or cold into the tissue to be ablated and/or into the body tissue containing the tissue to be ablated and/or on the basis of the thermal conductivity of the tissue to be ablated and/or the body tissue containing the tissue to be ablated and/or on the basis of the specific heat capacity of the tissue to be ablated and/or the body tissue containing the tissue to be ablated.

The thermal conductivity is used to take into account the diffusion of the heat or cold in the tissue to be ablated and/or the body tissue containing the tissue to be ablated. The specific heat capacity of, in particular, the tissue to be ablated can particularly be of importance to the effect that the tissue to be ablated should be heated slowly in order not to deposit too much heat locally in the tissue to be ablated. Too much heat could lead locally to an undesired premature carbonization of the tissue to be ablated. The carbonized tissue would then hinder the heat diffusion.

According to at least one example embodiment of the present invention, the perfusion measurement of the tissue to be ablated and/or the body tissue containing the tissue to be ablated is carried out preferably before or else during the ablation of the tissue of the body tissue to be ablated.

Particularly if the perfusion measurement takes place before the ablation, the temperature distribution in the tissue to be ablated and/or in the body tissue of the patient containing the tissue to be ablated can, according to at least one example embodiment of the present invention, be simulated over time for the treatment planning before the ablation is carried out.

For example, by varying the heat supply over time by means of one or more RF probes or RF needles in the tissue to be ablated, the temperature distribution in the tissue to be ablated and/or in the body tissue containing the tissue to be ablated can be simulated over time, more particularly on the basis of the flow speed of the blood through the tissue to be ablated and/or the body tissue containing the tissue to be ablated, the thermal conductivity of the tissue to be ablated and/or the body tissue containing the tissue to be ablated, and the specific heat capacity of the tissue to be ablated and/or the body tissue containing the tissue to be ablated, and the ablation can be planned on the basis of the simulation or the simulations such that it can be carried out as optimally as possible, i.e. with the best-possible expected treatment result.

According to at least one example embodiment of the present invention, image information of the tissue to be ablated and/or the body tissue of the patient containing the tissue to be ablated is obtained during the perfusion measurement and displayed on a viewing instrument. This makes it possible in the image information to distinguish between regions strongly perfused by blood and regions weakly perfused by blood, e.g. by means of corresponding highlighting in color on the basis of, for example, the flow speed. By way of example, the illustration can be brought about as a multiplanar reformation (MPR). If the display takes place during the intervention or the ablation, a medical practitioner performing the ablation can orient him/herself directly using the image information and can correspondingly control the supply of heat or cold.

According to at least one example embodiment of the present invention a device with a computational unit, which is designed in program-technical terms, i.e. has a computational program or a computer program implementing at least one of the above-described methods, in order to carry out at least one of the above-described methods.

According to at least one example embodiment of the present invention, the device can be a magnetic resonance scanner, an X-ray computed tomography scanner, an X-ray scanner, a C-arm scanner, an ultrasound scanner or a scanner for optical imaging, by means of which the perfusion measurement can be carried out and, for example, the progress of the intervention or the ablation can be followed.

According to at least one example embodiment of the present invention a data medium, with a computational program or computer program implementing at least one of the above-described methods and which computational program or computer program can be loaded from the data medium by a computational unit, which carries out at least one of the above-described methods when the computational program or computer program is loaded in the computational unit.

### BRIEF DESCRIPTION OF THE DRAWINGS

Example embodiments of the invention are illustrated in the attached schematic drawings, in which:

FIG. 1 shows a device in the form of a computed tomography scanner with a computational unit and

FIG. 2 shows three mutually orthogonal 2D slice images showing the liver of a patient from a perfusion measurement of the liver of the patient.

### DETAILED DESCRIPTION OF THE EXAMPLE EMBODIMENTS

Various example embodiments will now be described more fully with reference to the accompanying drawings in which only some example embodiments are shown. Specific structural and functional details disclosed herein are merely representative for purposes of describing example embodiments. The present invention, however, may be embodied in many alternate forms and should not be construed as limited to only the example embodiments set forth herein.

Accordingly, while example embodiments of the invention are capable of various modifications and alternative forms, embodiments thereof are shown by way of example in the drawings and will herein be described in detail. It should be understood, however, that there is no intent to limit example embodiments of the present invention to the particular forms disclosed. On the contrary, example embodiments are to cover all modifications, equivalents, and alternatives falling within the scope of the invention. Like numbers refer to like elements throughout the description of the figures.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items.

It will be understood that when an element is referred to as being “connected,” or “coupled,” to another element, it can be directly connected or coupled to the other element or intervening elements may be present. In contrast, when an element is referred to as being “directly connected,” or “directly coupled,” to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Spatially relative terms, such as “beneath”, “below”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, term such as “below” can encompass both an orientation of above and below. The device may be otherwise oriented (rotated 90 degrees or at other orientations) and the spatially relative descriptors used herein are interpreted accordingly.

Although the terms first, second, etc. may be used herein to describe various elements, components, regions, layers and/or sections, it should be understood that these elements, components, regions, layers and/or sections should not be limited by these terms. These terms are used only to distinguish one element, component, region, layer, or section from another region, layer, or section. Thus, a first element, component, region, layer, or section discussed below could be termed a second element, component, region, layer, or section without departing from the teachings of the present invention.

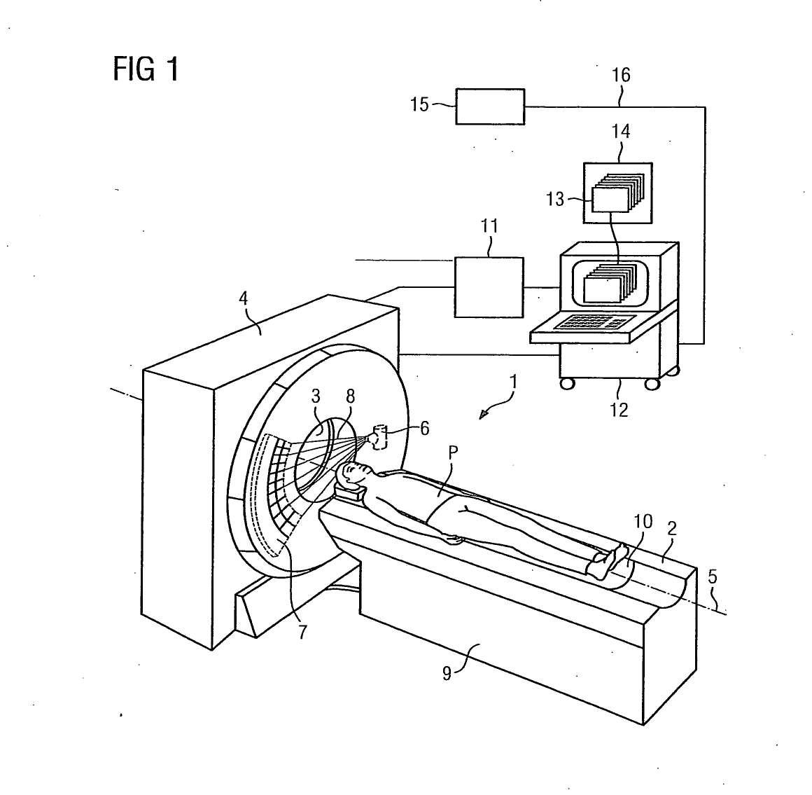

The computed tomography scanner 1 illustrated in FIG. 1 is only discussed in the following text and without loss of generality to the extent considered necessary for the understanding of example embodiments of the invention.

The computed tomography scanner 1 shown in FIG. 1 includes a patient couch 2 for supporting a patient P to be examined. The computed tomography scanner 1 furthermore includes a gantry 4 with a tube-detector system, rotatably mounted about a system axis 5. The tube-detector system includes an X-ray tube 6 and an X-ray detector unit 7 arranged mutually opposite to one another. During operation, X-ray radiation 8 is emitted in the direction of the X-ray detector unit 7 from the X-ray tube 6, and this X-ray radiation is detected by the X-ray detector unit.

The patient couch 2 includes a couch base 9, on which a patient support table 10 provided for actually supporting the patient P is arranged. The patient support table 10 can be adjusted relative to the couch base 9 such that the patient support table 10 with the patient P can be inserted into the opening 3 of the gantry 4 for recording 2D X-ray projections of the patient P, for example for a topogram or in a helical scan. A schematically illustrated image computer 11 of the computed tomography scanner 1 is used for the computational processing of the 2D X-ray projections, for example the production of a topogram or the reconstruction of a volume data record of a body region of the patient P on the basis of the 2D X-ray projections.

The computed tomography scanner 1 moreover includes a computational unit 12, which uses computational programs for operating and controlling the computed tomography scanner 1 and for planning examinations and treatments of patients can be carried out and is carried out. The computational unit 12 does not have to be embodied as a separate computational unit 12 in this case, but it can also be integrated into the computed tomography scanner 1.

In the case of the present example embodiment of the invention, the computational unit 12 may be used to simulate the temperature distribution in the tumor tissue to be ablated and in the liver tissue surrounding the tumor tissue to be ablated over time when the tumor tissue is heated over time during the ablation by means of an RF needle inserted into the tumor tissue; this may be done prior to an intervention, or for planning said intervention in the form of an RF ablation of tumor tissue in the liver of the patient P to be preferably followed by the computed tomography scanner 1.

To this end, a computational program, symbolically denoted by reference sign 13 in the figure, is loaded into the computational unit 12. The computational program 13 can be loaded into the computational unit 12 from a portable data medium, for example a CD 14, or from a server 15 via a network and implements a method in which the temperature distribution in the tumor tissue to be ablated and in the liver tissue containing or surrounding the tumor tissue to be ablated is determined taking into account at least one item of information relating to the blood flow over time in the tumor tissue and the liver tissue surrounding the tumor tissue. The computational program is embodied such that the computational program can both simulate the temperature distribution in a planning phase and also determine it in real-time during the ablation.

In the case of the present example embodiment of the invention, the information relating to the blood flow in the tumor tissue and the liver tissue surrounding the tumor tissue is the flow speed of the blood, which is obtained in a fashion known per se using a perfusion measurement of the liver of the patient P carried out using the computed tomography scanner 1. To this end, a certain amount of contrast agent is administered to the patient P in a defined fashion such that the occurrence and disappearance of contrast agent in the tumor tissue and the liver tissue surrounding the tumor tissue, both registered by the computed tomography scanner 1, can be used to establish the flow speed of the blood through the tumor tissue and the liver tissue surrounding the tumor tissue.

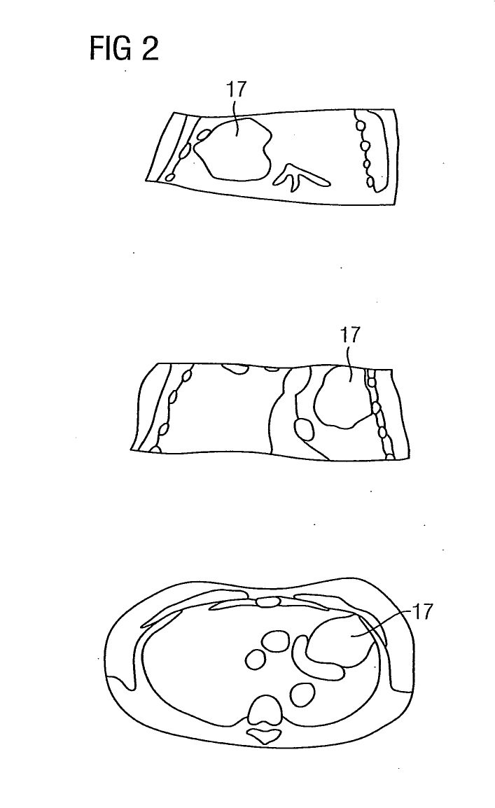

FIG. 2 shows, in an example fashion, three mutually orthogonal 2D slice images (direction of the views: lateral, anterior, and sagittal) showing the liver 17 of the patient P, which slice images originate from a perfusion measurement of the liver 17 of the patient P using the computed tomography scanner 1 from FIG. 1. The liver has regions with different perfusion and different flow speeds of the blood that cannot be identified in FIG. 2. The perfusion measurement is used to determine the respective flow speed of the blood for the various regions of the liver and the flow speed relevant for the tissue region where the ablation will be carried out is identified.

The determined and identified flow speed of the blood through the tumor tissue and the liver tissue surrounding the tumor tissue, and the heat supply into the tumor tissue over time by the RF needle are the input parameters for simulating the temperature distribution over time in the tumor tissue and the liver tissue surrounding the tumor tissue. Use can additionally be made of the thermal conductivity of the tumor tissue and/or the liver tissue, provided it is available, and the specific heat capacity of the tumor tissue and/or the liver tissue, provided it is available, for simulating the temperature distribution in the tumor tissue and the liver tissue surrounding the tumor tissue. Knowledge of the flow speed of the blood in particular and of the dissipation of the heat connected thereto allow the temperature distribution in the tumor tissue and the liver tissue surrounding the tumor tissue to be simulated relatively precisely over time as a function of the heat supply over time, and thus allow the ablation to be planned.

The temperature distribution in the tumor tissue and the liver tissue surrounding the tumor tissue can also be determined according the described method while the ablation is carried out. In the process, a parallel perfusion measurement of the liver tissue can establish the current flow speed of the blood during the ablation and hence the temperature distribution in the tumor tissue and the liver tissue surrounding the tumor tissue can be determined on the basis of the current established value of the flow speed.

Moreover, the device does not necessarily require a computed tomography scanner for monitoring the ablation and for measuring the perfusion in the liver tissue. Rather, the imaging scanner can also be a magnetic resonance scanner, an X-ray scanner; a C-arm scanner or an ultrasound scanner.

Furthermore, it is possible to use a plurality of RF probes or RF needles for supplying the heat into the tumor tissue, the respective heat supply of which has to be accordingly taken into account during the simulation or determination of the temperature distribution in the tissue to be ablated and the liver tissue surrounding the tissue to be ablated.

The simulation and determination of the temperature distribution is not restricted to tumor tissue in the liver, but it can also be used in other tissues or organs, e.g. the kidneys.

The computational unit need not necessarily be connected to the computed tomography scanner for the simulation provided that the computational unit has available the established flow speed of the blood and the data relating to the heat supply into the tumor tissue over time for simulating the temperature distribution over time.

The ablation itself can also be carried out by means of ablation methods that differ from RF ablation, for example by means of cryoablation.

What is claimed is:

1. A method for determining a temperature distribution in at least one of a tissue to be ablated and in a body tissue of a patient containing the tissue to be ablated, the method comprising:
determining at least one item of information relating to a blood flow associated with at least one of in the tissue to be ablated and in the body tissue containing the tissue to be ablated, such that the item of information originates from a perfusion measurement of at least one of the tissue to be ablated and the body tissue containing the tissue to be ablated; and
determining the temperature distribution at least one of in the tissue to be ablated and in the body tissue containing the tissue to be ablated taking into account the at least one item of information relating to the blood flow.

2. The method as claimed in claim 1, further comprising:
establishing, within the scope of the perfusion measurement, at least one of (1) a flow speed of the blood of the patient flowing through at least one of the tissue to be ablated and the body tissue containing the tissue to be ablated and (2) a blood volume, which flows through at least one of the tissue to be ablated and the body tissue containing the tissue to be ablated over time.

3. The method as claimed in claim 1, wherein the temperature distribution in at least one of the tissue to be ablated and in the body tissue containing the tissue to be ablated is further determined based on at least one of,
an ingress of heat or cold at least one of into the tissue to be ablated and into the body tissue containing the tissue to be ablated,
a thermal conductivity of at least one of the tissue to be ablated and the body tissue containing the tissue to be ablated, and
a specific heat capacity of at least one of the tissue to be ablated and the body tissue containing the tissue to be ablated.

4. The method as claimed in claim 1, wherein the perfusion measurement is one of performed before and during the ablation of the tissue of the body tissue to be ablated.

5. The method as claimed in claim 1, wherein the temperature distribution is simulated before the ablation is carried out.

6. The method as claimed in claim 1, further comprising:
obtaining image information associated with one of the tissue to be ablated and the body tissue of the patient containing the tissue to be ablated during the perfusion measurement; and
displaying the image information on a viewing instrument.

7. A device including a computational unit, wherein the device includes code segments that when executed by the computational unit cause the device to perform the steps of the method as claimed in claim 1.

8. The device as claimed in claim 7, wherein the device is one of a magnetic resonance scanner, an X-ray computed tomography scanner, an X-ray scanner, a C-arm scanner, an ultrasound scanner and a scanner for optical imaging.

9. A data medium on which a program to implement the method as claimed in claim 1 is stored such that when the program is executed by a computational unit, the computational unit performs the method of claim 1.

10. The method as claimed in claim 2, wherein the temperature distribution in at least one of the tissue to be ablated and in the body tissue containing the tissue to be ablated is further determined based on at least one of,
an ingress of heat or cold at least one of into the tissue to be ablated and into the body tissue containing the tissue to be ablated,
a thermal conductivity of at least one of the tissue to be ablated and the body tissue containing the tissue to be ablated, and
a specific heat capacity of at least one of the tissue to be ablated and the body tissue containing the tissue to be ablated.

11. The method as claimed in claim 2, wherein the perfusion measurement is one of performed before and during the ablation of the tissue of the body tissue to be ablated.

12. The method as claimed in claim 2, wherein the temperature distribution is simulated before the ablation is carried out.

13. The method as claimed in claim 2, further comprising:
obtaining image information associated with one of the tissue to be ablated and the body tissue of the patient containing the tissue to be ablated during the perfusion measurement; and
displaying the image information on a viewing instrument.

14. The device as claimed in claim 7, wherein the temperature distribution in at least one of the tissue to be ablated and in the body tissue containing the tissue to be ablated is further determined based on at least one of,
an ingress of heat or cold at least one of into the tissue to be ablated and into the body tissue containing the tissue to be ablated,
a thermal conductivity of at least one of the tissue to be ablated and the body tissue containing the tissue to be ablated, and
a specific heat capacity of at least one of the tissue to be ablated and the body tissue containing the tissue to be ablated.

15. The device as claimed in claim 7, wherein the perfusion measurement is one of performed before and during the ablation of the tissue of the body tissue to be ablated.

16. The device as claimed in claim 7, wherein the temperature distribution is simulated before the ablation is carried out.

17. The device as claimed in claim 7, wherein the method further comprises:
obtaining image information associated with one of the tissue to be ablated and the body tissue of the patient containing the tissue to be ablated during the perfusion measurement; and
displaying the image information on a viewing instrument.
